(19) United States
(12) Patent Application Publication
(10) Pub. No.: US 2008/0108677 A1
(43) Pub. Date: May 8, 2008

(54) Thioxothiazolidinone Compounds For Use As Pharmaceuticals

(75) Inventors: Mikael Bjorklund, Helsinki (FI); Erkki Koivunen, Helsinki (FI)

Correspondence Address: BIRCH STEWART KOLASCH & BIRCH, PO BOX 747, FALLS CHURCH, VA, 22040-0747, US

(73) Assignee: Karyon-CTT Ltd, Helsinki (FI)

(21) Appl. No.: 11/661,505

(22) PCT Filed: Aug. 30, 2005
(86) PCT No.: PCT/FI05/50305

§ 371 Date: Apr. 23, 2007

(30) Foreign Application Priority Data
Aug. 30, 2004 (FI) 20041129

(51) Int. Cl. A61K 31/427; C07D 277/04; A61P 35/00

(52) U.S. Cl. 514/369; 548/183

(57) ABSTRACT

The present invention relates to thioxothiazolidinone compounds for use as pharmaceuticals, to pharmaceutical compositions comprising these compounds, and to the use of said small-molecule compounds for the manufacture of pharmaceutical compositions for the treatment of conditions dependent on leukocyte cell migration, such as leukaemia and inflammatory diseases. Said compounds inhibit leukaemia cell migration by stabilizing the active conformation of the α_{M} integrin I domain.

### FIELD OF THE INVENTION

The present invention relates to thioxothiazolidinone compounds for use as pharmaceuticals, to pharmaceutical compositions comprising these compounds, and to the use of said small-molecule compounds for the manufacture of pharmaceutical compositions for the treatment of conditions dependent on leukocyte cell migration, such as leukaemia, other malignancies/cancers, and inflammatory diseases. Said compounds inhibit leukaemia cell migration by stabilizing the active conformation of the α_{M} integrin I domain.

### BACKGROUND OF THE INVENTION

Integrins are a large family of heterodimeric cell surface receptors intimately involved in cell adhesion, migration and signalling (Hynes, 2002). Studies with the α_{V}β₃ integrin and the leukocyte-specific α_{L}β₂ and α_{N}β₂ integrins have been instrumental in understanding the integrin structure and function (Beglova et al., 2002; Luo et al., 2004; Ruoslahti, 1996; Salas et al., 2004; Shimaoka et al., 2003; Xiong et al., 2001; Xiong et al., 2002). Some integrins, including the β₂ integrins contain an inserted (I) domain in the a subunit as the major ligand-binding site, whereas those lacking the I domain use the α and β chains to form the ligand-binding pocket (Hynes, 2002). There is a considerable interest in compounds affecting integrin function(s) due to the enormous clinical potential in the treatment of various inflammatory conditions, cancer and other diseases. Consequently, many small molecule inhibitors to the α_{L} integrin I domain and other integrins have been identified (Goodman et al., 2002; Last-Barney et al., 2001; Liu et al., 2001; Weitz-Schmidt et al., 2001). The αintegrin antagonists act allosterically by binding underneath the C-terminal α helix of the I domain and prevent ligand binding by stabilizing the low-affinity conformation of the I domain (Last-Barney et al., 2001; Liu et al., 2001; Weitz-Schmidt et al., 2001). Another class of β₂ integrin selective small molecules bind to the integrin β₂ subunit I-like domain and prevent the activation of the α_{L} I domain, but at the same time they induce the active conformation of the I-like domain and the stalk domains (Shimaoka et al., 2003). Additionally, two small-molecule antagonists of α_{M}β₂ integrin have been recently identified (Bansal et al., 2003). These compounds inhibit complement protein iC3b, but not intercellular adhesion molecule (ICAM)-1 binding to the α_{M}β₂ integrin. They also block leukocyte adhesion to fibrinogen and adhesion-associated oxidative burst (Bansal et al., 2003).

Using phage display, we previously identified a peptide ligand ADGACILWMDDGWCGAAG (DDGW) binding to the αand α_{L} integrin inserted (I) domains. The DDGW peptide mimicked the integrin-binding sequence of the latent matrix metalloproteinase (MMP)-2 and -9 and inhibited leukocyte migration in vitro and in vivo (Stefanidakis et al., 2003; Stefanidakis et al., 2004). Although DDGW and other phage display-derived peptides function well in vitro and in animal models (Koivunen et al., 1999; Koivunen et al., 2001; Pasqualini et al., 2000), rapid clearance and susceptibility to proteolysis may limit the use of peptides in a clinical setting. Hence, it is necessary to convert the phage display peptides into peptidomimetics or screen for small-molecule libraries to obtain pharmacologically suitable drug leads (Hyde-DeRuyscher et al., 2000; Kay et al., 1998; Ripka and Rich, 1998). Here, by using the DDGW peptide we identify a novel class of compounds that inhibit α_{M}β₂ integrin-mediated migration of leukemic cells by locking the α_{M} I domain into an active conformation.

### SUMMARY OF THE INVENTION

We previously identified a peptide ligand to the α_{M} integrin inserted (I) domain by phage display. This peptide inhibited interaction between promatrix metalloproteinase-9 (proMMP-9) and α_{M}β₂ integrin, and suppressed leukocyte migration. By screening a combinatorial library with the aid of this peptide, we have now identified novel small-molecule ligands to α_{M}β₂ integrin. Strikingly, these compounds stabilize α_{M} I domain binding to its ligands, proMMP-9 and fibrinogen. The compounds did not enhance or inhibit primary adhesion, but induced resistance of α_{M}β₂ integrin-expressing cells to detachment by EDTA treatment. In addition, the compounds potently inhibited migration of leukemic cells independently on MMP-9 activity indicating that the migration defect was primarily caused by the inability of the cells to detach. Such small-molecule α_{M}β₂ integrin ligands have utility in treatment of leukaemia, other malignancies/cancer and inflammatory diseases characterized by active α_{M}β₂ integrin-dependent cell migration.

Consequently, the invention is directed to compounds of formula I

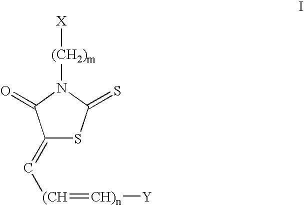

wherein m is 0, 1 or 2;

X is H, cycloalkyl or phenyl, which is unsubstituted or substituted with one or more substituents selected from the group consisting of lower alkyl, hydroxy and halogen;

n is 0 or 1;

Y is phenyl, furanyl, indole or pyrrole, which all may be substituted with one or more substituents selected from the group consisting of lower alkyl, lower alkoxy, halogen, (3,5-dimethylphenoxy)propoxy, and phenyl, wherein phenyl may be further substituted with one or more halogen atoms, nitro, amino or carboxyl groups; for use as pharmaceuticals.

The invention is also directed to pharmaceutical compositions comprising a thioxothiazolidinone compound of formula I

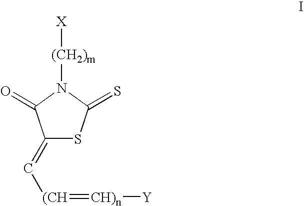

wherein m is 0, 1 or 2;

X is H, cycloalkyl or phenyl, which is unsubstituted or substituted with one or more substituents selected from the group consisting of lower alky, hydroxy and halogen;

n is 0 or 1;

Y is phenyl, furanyl, indole or pyrrole, which all may be substituted with one or more substituents selected from the group consisting of lower alkyl, lower alkoxy, halogen, (3,5-dimethylphenoxy)propoxy, and phenyl, wherein phenyl may be further substituted with one or more halogen atoms, nitro, amino or carboxyl groups; and a pharmaceutically acceptable carrier.

A further object of the invention is the use of a thioxothiazolidinone compound of the formula I as defined above for the manufacture of a pharmaceutical composition for the treatment of conditions dependent on leukocyte cell migration, such as leukaemia and inflammatory conditions.

Further objects of the invention are the corresponding methods, i.e. a method for therapeutic or prophylactic treatment of conditions dependent on leukocyte cell migration, and a method for therapeutic or prophylactic treatment of leukaemia, other malignancies/cancers, or inflammatory conditions, wherein at least one thioxothiazolidinone compound of the formula I as defined above is administered to a mammal in need of such treatment.

Within this disclosure, “lower” in connection with alkyl or alkoxy refers to substituents having 1-6, preferably 1-4, carbon atoms. Lower alkyl is preferably methyl or ethyl. As it comes to lower alkoxy, methoxy is preferred. Cycloalkyl has preferably 3-8, even more preferably 4-6 carbon atoms, and is preferably cyclohexyl.

Halogen may be fluorine, chlorine, bromine, or iodine.

Preferred thioxothiazolidinone compounds are compounds of formula I, wherein m is 0, X is unsubstituted phenyl or phenyl ortho-substituted with methyl, n is 1, and Y is unsubstituted phenyl. As regards stereochemistry, compounds having Z-configuration are preferred but also E-configuration is possible.

Even more preferred thioxothiazolidinone compounds are selected from the group consisting of

(E)-5-(4-(3-(3,5-dimethylphenoxy)propoxy)-3-methoxybenzylidene)-3-ethyl-2-thioxothiazolidin-4-one (referred herein as IMB-2);

(Z)-3-benzyl-5-((5-(3-nitrophenyl)furan-2-yl)methylene)-2-thioxothiazolidin-4-one (IMB-6); and

(5Z)-5((E)-3-phenylallylidene)-2-thioxo-3-o-tolylthiazolidin-4-one (IMB-10).

In this specification, unless otherwise indicated, terms such as “compounds of formula I” embrace, if appropriate, the compounds in salt form as well as in free base (or in free acid, or in free acid or base) form. Only pharmaceutically applicable salts are included.

The invention is herein below described in more detail referring to the accompanied figures.

### BRIEF DESCRIPTION OF THE FIGURES

FIG. 1. (A) Chemical structures of the α_{M}β₂/iC3b interaction inhibiting compounds (Bansal et al., 2003) and (B) the preferred α_{M} I domain-binding compounds identified in this study and their inhibitory activities in the DDGW phage-binding assay. The IC₅₀ value for DDGW-α_{M} I domain interaction is calculated as the mean of three independent experiments in triplicates.

FIG. 2. (A) Dose-dependent inhibition of DDGW-phage binding by soluble DDGW peptide or the compounds. (B) Binding of α_{M} I domain-GST fusion to intact proMMP-9 and the catalytically inactive, C terminally truncated proMMP-9-ΔHC-E⁴⁰²Q. Soluble GST alone does not bind to the MMP-9. Binding of α_{M} I domain to proMMP-9-ΔHC-E⁴⁰²Q (C) and fibrinogen (D) in the presence of DDGW peptide (100 μM), chemicals (50 μM) or vehicle (DMSO, 1%). Bound protein was detected with anti-GST antibody. Data shown is mean±SD from triplicate samples. The experiments were repeated three times.

FIG. 3. Effect of divalent cations in the α_{M} I domain binding to proMMP-9-ΔHC-E⁴⁰²Q (A) and fibrinogen (B) in the presence or absence of 10 μM IMB-10 or DMSO as vehicle.

FIG. 4. (A) Antibody binding to the recombinant α_{M} I domain-GST fusion or GST alone in the presence of DDGW peptide (100 μM), chemical competitors (50 μM) or vehicle (DMSO). Bound antibody was detected with peroxidase-conjugated anti-mouse antibody. The epitope for OKM10 antibody is located outside the α_{M} integrin I domain. (B) Antibody binding to purified immobilized α_{M}β₂ integrin. The chemicals were used at a 25 μM concentration. Data shown is mean±SD from triplicate samples. The experiments were repeated two to four times.

FIG. 5. (A) Adhesion of phorbol-ester activated THP-1 cells on plastic in the presence of 25 μM compounds or vehicle (DMSO) or without phorbol ester activation (no act.). The cells were allowed to attach overnight. Cells remaining attached after washing with PBS or 2.5 mM EDTA in PBS were photographed. Bar 200 μM. (B) The EDTA washed, plastic-adherent THP-1 cells were quantitated with a phoshatase assay. (C) Adhesion of THP-1 cell to fibrinogen in the presence or absence of the competitors (25 μM). The cells were stimulated with 50 nM PDBu and allowed to adhere for 30 minutes. After washing with PBS or 2.5 mM EDTA, the adherent cells were quantitated by comparison of the phosphatase activity of known amounts of cells. The assays were conducted in triplicates and repeated at least three times.

FIG. 6. (A) Migration of THP-1 cells on a synthetic LLG-C4-GST coating in the presence of 25 μM compounds or 100 μM soluble LLG-C4 peptide. Migration on GST alone is shown as a control. (B) Migration of THP-1 and OCI-AML-3 cells on fibrinogen in the presence or absence of the competitors. The cells were activated with 40 nM PDBu and allowed to migrate for 16 hours. The gelatinase-selective small molecule inhibitor InhI (50 μM) is shown as a control to evaluate the level of gelatinase-dependent migration. (C) Migration of HT1080 fibrosarcoma cells on serum coated transwells in the presence of competitors. The results are representative from two to three experiments. (D) Pericellular proteolysis of urokinase-plasminogen activator receptor (uPAR). OCI-AML-3 cells were stimulated with PDBu or left untreated and cultured for 48 hours in the presence of 20 μM IMB-10, the gelatinase-selective inhibitor (InhI) or vehicle (DMSO). uPAR and the cleaved form of uPAR D2+3 was detected with western blotting from detergent-enriched cell lysates.

FIG. 7. The percentage indicates inhibition of DDGW-phage binding at a 10 μM concentration.

FIG. 8. IMB-10 inhibits leukocyte recruitment in vivo. Thioglycollate (TG) was used to induce peritonitis in mice. PBS was used as a control. The mice received IMB-8 or IMB-10 as an intravenous injection (i.v.). Cells migrated to the peritoneal cavity were collected after 3 or 24 h and counted. Data shown is mean±SEM (n=5). Statistical difference between the TG injected mice was studied with ANOVA, and the observed differences between the groups were compared using the Bonferroni test. An asterisk indicates statistical significance (p<0.01).

### DETAILED DESCRIPTION OF THE INVENTION

We have identified a novel class of small molecule ligands, which stabilize the active conformation of the α_{M} I domain. These compounds are structurally distinct from the previously characterized α_{M} and α_{L} I domain antagonists (Bansal et al., 2003; Kelly et al., 1999; Liu et al., 2001; Shimaoka et al., 2003; Weitz-Schmidt et al., 2001). IMB-10, the most potent of the identified compounds, increased the binding of recombinant α_{M} I domain to its ligands proMMP-9 and fibrinogen. Remarkably, IMB-10 also made α_{M}β₂ integrin-expressing cells highly resistant to the effect of the cation chelator EDTA, consistent with the chemical's role as a stabilizer of the active α_{M} I domain. The IMB-10 compound was also a highly potent inhibitor of α_{M}β₂ integrin-mediated leukemia cell migration.

Although the compounds of formula I were identified as inhibitors of DDGW-peptide bearing phage binding to the α_{M} I domain, they failed to inhibit the proMMP-9/α_{M} I domain interaction. This difference may be traced to the fact that we initially identified the DDGW peptide and the proMMP-9/α_{M}β₂ integrin interaction in a calcium-containing buffer (Stefanidakis et al., 2003). Our data suggests that DDGW peptide preferentially binds to the closed conformation of the I domain, whereas proMMP-9 binds both open and closed conformation, although preferring the open conformation. The inability of IMBs to inhibit proMMP-9 interaction with the I domain clearly indicates that the IMBs and DDGW bind to different sites. Most small-molecule compounds are uncharged and thus may not occupy the same binding site as a charged peptide or protein ligand. Such charged sequence motifs are typical for integrin ligands (Arnaout et al., 2002). In accordance with this, all small-molecule ligands of the α_{L} I domain are allosteric antagonists (Kelly et al., 1999; Last-Barney et al., 2001; Liu et al., 2001; Weitz-Schmidt et al., 2001). Indeed, it may be difficult to identify small-molecular compounds that directly mimic the action of such charged peptide ligands. In this respect, phage display can provide novel ligands to sites that cannot be well occupied by small-molecule compounds used in high-throughput screenings. Apparently, phage display can reveal biologically important sites, which would remain unnoticed in small-molecule screenings.

The activation state of the recombinant α_{M} and α_{L} I domains can be regulated by a site distinct from the ligand-binding metal ion dependent adhesion site (MIDAS) (Kallen et al., 1999; Xiong et al., 2000). A single point mutation Ile³¹⁶→Gly near the C terminus of the α_{M} I domain locks the I domain in the constitutively active, open conformation (Xiong et al., 2000). In the closed α_{M} I domain structure this Ile³¹⁶ residue lies in a hydrophobic socket that is in a nearly analogous location, where lovastatin binds in the α_{L} I domain (Kallen et al., 1999). In the absence of a ligand, a closely balanced equilibrium between the open and closed conformation of the α_{M} I domain is evident, with 10-12% of the protein present in the open conformation. Instead, the α_{L} I domain is exclusively in the closed conformation (McCleverty and Liddington, 2003). Thus there is a high possibility to obtain ligands for the active conformation of the α_{M} I domain.

Although IMB-10 did not inhibit proMMP-9 binding, it was far more potent inhibitor of leukemia cell migration than the DDGW peptide. The IMB-10 interfered only with β₂ integrin-dependent migration, as there was no effect on the migration of HT1080 fibrosarcoma cells, which express other integrins. The ability of IMB-10 to inhibit cell migration on fibrinogen appears to be independent on gelatinase activity, as the small-molecule gelatinase inhibitor (InhI) did not block cell migration. Furthermore, IMB-10 did not inhibit pericellular gelatinase-dependent proteolysis of uPAR. Collectively our data indicates that the inhibition of leukemia cell migration by IMB-10 is caused primarily due to enhanced adhesion and not by inhibition of integrin-regulated gelatinase activity. Too strong adhesion inhibits cell motility (Palecek et al., 1997) and this phenomenon may be utilized to develop antagonists of cell migration as exemplified by our studies.

### Experimental

Screening of the compound library. A combinatorial library of 10 000 small molecules was purchased from ChemBridge (San Diego, Calif.). A competition assay with the DDGW peptide bearing phage was set up by immobilizing 20 ng/well recombinant α_{M} I domain-GST fusion in 96-well plates (Michishita et al., 1993). The compounds were used in pools comprising eight compounds, each at a 5 μM concentration and DMSO at a 1.25% concentration. After preincubation of the compounds in the wells, DDGW phage was added (3×10⁸ transducing units/well). As controls, soluble DDGW peptide and a control peptide, and an unrelevant phage were included in every plate. Phage binding was detected with an anti-phage antibody as described (Bjorklund et al., 2004). Pools with inhibitory activity were re-tested as single compounds. The DDGW-phage inhibiting activity of these hits at a 10 μM concentration is shown in supplementary data. To calculate the IC₅₀ values for the compounds, dilution series from the DDGW peptide and compounds were made (10 μM to 150 nM) and DDGW phage binding was measured. No inhibitor (DMSO as a vehicle) was used as 100% binding after subtracting the background value obtained with an irrelevant control phage.

proMMP-9 and fibrinogen binding to the α_{M} integrin I domain. The catalytically inactive proMMP-9-ΔHC-E⁴⁰²Q mutant was prepared via site-directed mutagenesis from the wild-type proMMP-9-ΔHC and was purified using gelatin-sepharose (Bjorklund et al., 2004). The resulting MMP-9 with this mutation is structurally identical to the wild type protein (Rowsell et al., 2002). ProMMP-9-ΔHC-E⁴⁰²Q, intact proMMP-9 or fibrinogen (100 ng/well) was coated on microtiter wells in TBS followed by saturation of the wells with 1% BSA in PBS/0.05% Tween20. Soluble α_{M} integrin I domain-GST fusion (2.5 μg/ml) was added in the presence or absence of peptides or compounds in 0.1% BSA/TBS/0.05% Tween20/1 mM CaCl₂/1 mM MgCl₂, and incubated for one hour. In some experiments 10 mM CaCl₂ or 10 mM MgCl₂ were used instead of 1 mM CaCl₂/1 mM MgCl₂. Bound GST fusion was detected with anti-GST antibody (1:2000 dilution) and peroxidase-conjugated anti-goat antibody (1:2000 dilution).

Inhibition of antibody binding to the integrins. The α_{M} I domain or purified α_{M}β₂ integrin (50 ng/well) were coated on microtiter wells followed by saturation of the wells with 1% BSA in PBS/0.05% Tween20. The DDGW peptide or the compounds at concentrations indicated were preincubated for 30 minutes with the integrin, followed by addition of the antibodies LM2/1, MEM170, OKM-10, IB4 or a control IgG at a 1 μg/ml final concentration. The antibodies LM2/1 and MEM170 recognize the α_{M} integrin I domain, whereas the epitope for OKM-10 is located outside the α_{M} I domain (Koivunen et al., 2001; Li et al., 1995; Stefanidakis et al., 2003). The antibodies were incubated for 45 minutes followed by detection of the bound antibodies with a peroxidase conjugated anti-mouse antibody.

Cell culture. Human monocytic leukemia THP-1, OCI-AML-3 acute myeloid leukemia and human HT1080 fibrosarcoma cells were cultured as described (Koivunen et al., 1999; Koivunen et al., 2001; Stefanidakis et al., 2003). The cell viability was measured using an 3-[4,5-dimethylthiazol-2-yl]-2,5-diphenyl tetrazolium bromide assay as described (Bjorklund et al., 2004).

Cell adhesion and migration. Cell adhesion was performed as described (Koivunen et al., 2001). Briefly, THP-1 cells (50 000/well) were stimulated with 50 nM PDBu (4β-phorbol-12,13-dibutyrate, SigmaAldrich) and allowed to bind to the fibrinogen coated microtiter wells (10 μg/ml) in the presence or absence of compounds or DMSO in serum-free RPMI medium containing 0.1% BSA. Non-adherent cells were removed by gentle washing with PBS or 2.5 mM EDTA in PBS, and adherent cells were quantitated with a phosphatase assay (Koivunen et al., 2001). Alternatively, THP-1 cells were stimulated with 20 nM PDBu and allowed to adhere on uncoated plastic overnight. Nonadherent cells were removed by washing with PBS followed by six washes with 2.5 mM EDTA in PBS. Cell migration was done using transwells (5 μm pore size) coated with 40 μg/ml LLG-C4-GST or fibrinogen (Koivunen et al., 2001). THP-1 and OCI-AML-3 cells (50 000 cells/100 μl) were allowed to migrate overnight in 10% FBS/RPMI medium in the presence or absence of peptides or compounds and stimulating cell migration with 40 nM PDBu. Gelatinase-selective inhibitor I (InhI) at a 50 μM concentration (Calbiochem) was used to evaluate the level of gelatinase-dependent migration on fibrinogen. Migrated cells were stained with crystal violet and counted (Koivunen et al., 2001). Migration of HT1080 cells on serum coated transwells was conducted as described (Koivunen et al., 1999).

Cellular cleavage of the urokinase-plasminogen activator receptor. Pericellular gelatinase-dependent uPAR cleavage was performed as described (Bjorklund et al., 2004). Briefly, OCI-AML-3 cells were stimulated with PDBu or left untreated and cultured for 48 hours in the presence of 20 μM IMB-10, the gelatinase-selective inhibitor (InhI) or vehicle (DMSO). uPAR and the cleaved form of uPAR D2+3 was detected with western blotting using a polyclonal antibody to uPAR (399R, American Diagnostica).

Neutrophil emigration in vivo. The mouse experiments were approved by the ethical committee for the animal experiments at the University of Helsinki. Inflammation was induced in BALB/cOlaHsd female maintained in the Viikki Laboratory Animal Centre by injecting 1 ml 4% thioglycollate broth in PBS i.p. followed by intravenous injections of chemicals (200 μl at a 20, 5 or 1 μg/ml concentration) or PBS after five minutes. Cells emigrated in the peritoneum after 3 or 24 h were counted using a hemocytometer. Statistical significance between groups in the peritonitis model was calculated with one-way ANOVA and found differences were analyzed by Bonferroni pairwise multiple comparison tests. Results with p<0.01 were deemed significant.

### Results

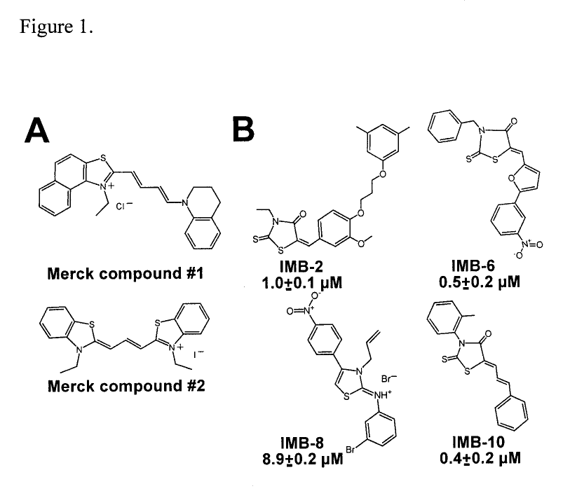

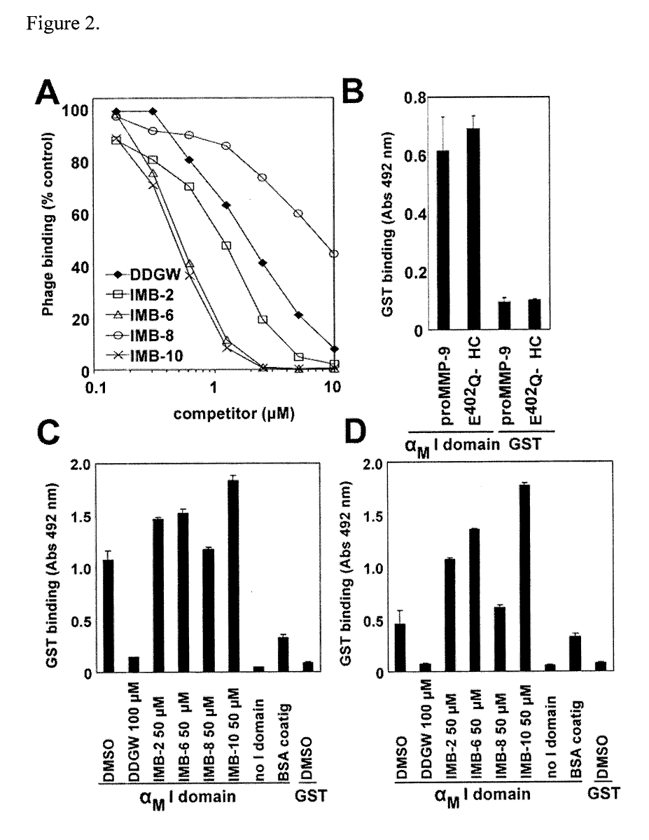

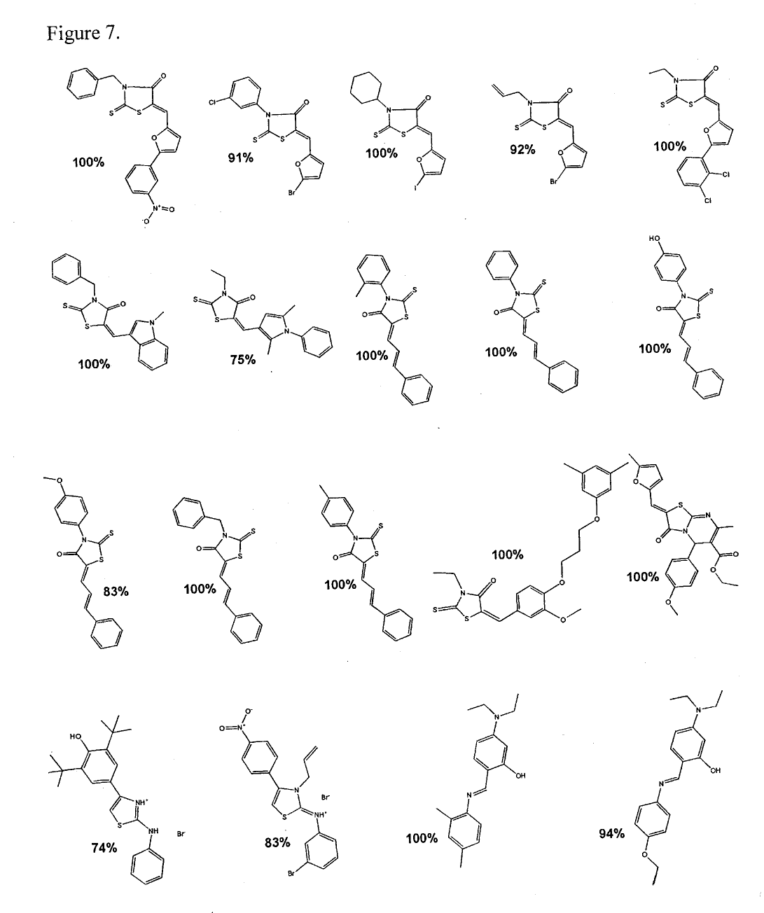

We screened a combinatorial library in order to identify small-molecules, which would block leukemia cell migration by inhibiting the previously characterized interaction between proMMP-9 and the leukocyte integrins. Several compounds were identified in this screen, most of which had a common thioxothiazolidinone substructure (FIG. 1 and FIG. 7). The structures of iC3b/α_{M}β₂ interaction blocking compounds (Bansal et al., 2003) are shown for comparison (FIG. 1A). The newly identified compounds specifically inhibited DDGW-phage binding to the α_{M} I domain, but had no effect on binding of CRV-peptide to the C terminal domain of MMP-9 (Bjorklund et al., 2004) (data not shown). Representative compounds IMB-2, -6, -8 and -10 (FIG. 1B) were tested to measure their IC₅₀ values for the inhibition of DDGW-phage binding to the α_{M} I domain (FIG. 2A). The best compound, IMB-10 had an IC₅₀ value of 0.4±0.2 μM, six-fold better than the IC₅₀ value 2.6±0.5 μM for the soluble DDGW peptide. These compounds also inhibited DDGW phage binding to the α_{L} I domain (not shown), but this interaction is significantly weaker than the binding to the α_{M} I domain (Stefanidakis et al., 2003).

We next evaluated the effect of the compounds on proMMP-9 binding to α_{M} I domain. The α_{M} I domain binding site in MMP-9 is mapped to the DDGW-like negatively charged sequence present in the catalytic domain of MMP-9. However, it is unknown if the I domain binding activity is dependent on the catalytic activity of the MMP-9 as gelatinase-selective inhibitors block this interaction (Stefanidakis et al., 2003). To resolve this question, we prepared a catalytically inactive proMMP-9 mutant lacking the collagen V-like hinge region and the C-terminal domain (proMMP-9-E⁴⁰²Q-ΔHC). Strong binding of soluble α_{M} I domain-GST to immobilized proMMP-9-E⁴⁰²Q-ΔHC was observed and the level of binding was comparable to intact proMMP-9 (FIG. 2B). GST alone did not bind proMMP-9. The interaction of α_{M} I domain to proMMP-9-E⁴⁰²Q-ΔHC was inhibited by the DDGW peptide (FIG. 2C). Surprisingly, although identified by the DDGW phage assay, none of the chemicals inhibited binding of α_{M} I domain to proMMP-9-E⁴⁰²Q-ΔHC (FIG. 2C) or proMMP-9 (not shown) at a 50 μM concentration. Instead, the α_{M} I domain binding activity was markedly enhanced by the IMBs. The level of activation correlated with their IC₅₀ values in the phage assay. The α_{M} I domain binding to fibrinogen was enhanced by even more in the presence of the chemicals (FIG. 2D). This unexpected behaviour suggested that DDGW and the identified chemicals do not compete for the same binding site and that the IMBs might actually stabilize the active conformation of the α_{M} I domain. The compounds of formula I thus show a novel activity causing enhanced ligand-binding activity in contrast to the previously identified small-molecule ligands to the α_{L} I domain, which inhibit ligand binding.

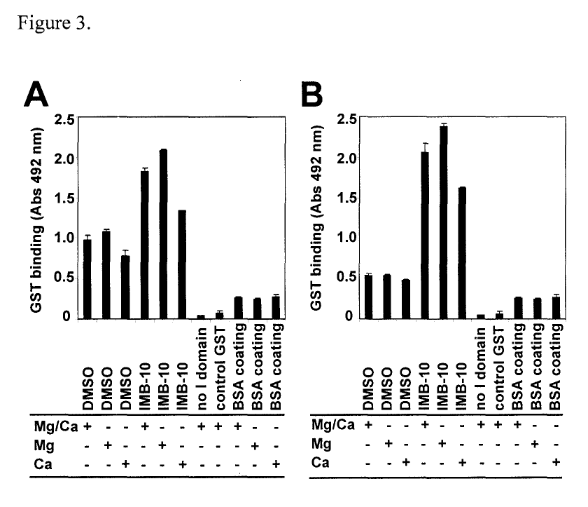

To investigate the possible stabilization of the α_{M} I domain, binding assays were conducted in the presence of 10 mM Ca²⁺ or 10 mM Mg²⁺ to maintain the I domain in the inactive or active conformation, respectively. A strong binding of α_{M} I domain to proMMP-9-E⁴⁰²Q-ΔHC was observed in the presence of magnesium and IMB-10 at a 10 μM concentration, whereas calcium nearly completely antagonized the effect of IMB-10 (FIG. 3A). Binding in the presence of equimolar concentrations of Ca²⁺ and Mg²⁺ was intermediate to that of Ca²⁺ and Mg²⁺ alone. Similar effect was obtained with α_{M} I domain binding to fibrinogen (FIG. 3B).

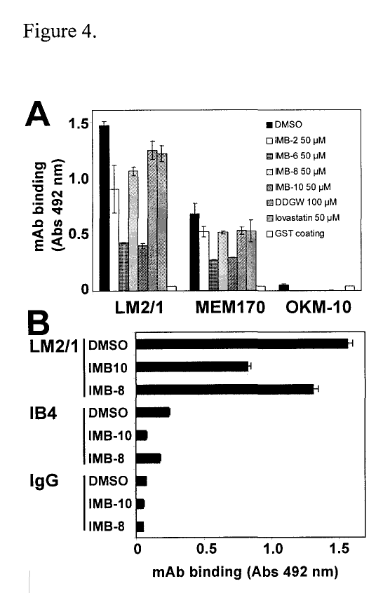

We next evaluated the ability of DDGW peptide and the chemicals to compete with antibody binding to immobilized α_{M} I domain-GST fusion. The IMBs inhibited mAb LM2/1 binding in accordance their DDGW-peptide inhibitory potency, with 75% inhibition by IMB-6 and -10 at a 50 μM concentration (FIG. 4A). The compounds also inhibited the binding of another α_{M} I domain specific antibody MEM170. The DDGW peptide or lovastatin had only a marginal effect on antibody binding. The IMBs also inhibited LM 2/1 antibody binding to purified α_{M}β₂. Binding of mAb IB4, was also inhibited by IMB-10. The epitope of this antibody is located in the β₂ I-like domain (FIG. 4B). Binding of TS1/22 and MEM83 antibodies to α_{L} I domain were similarly affected by the IMB-10 chemical, but not by IMB-8. Before conducting cell-based experiments, we tested possible toxicity of the chemicals. The compounds were incubated with THP-1 monocytic leukemia and OCI-AML-3 acute myeloid leukemia cells for 48 h in serum-containing cell culture medium. The IMB-6 was toxic to both cell lines apparently due to low solubility, whereas IMB-2, IMB-8 and IMB-10 had no significant effect on cell proliferation at a 50 μM concentration (data not shown).

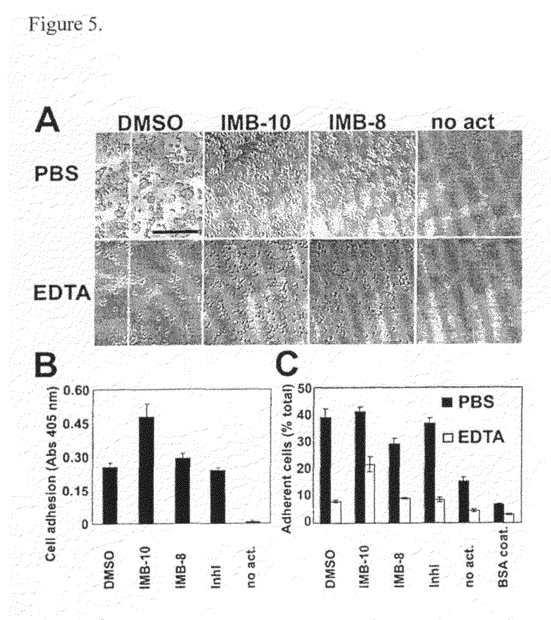

Adhesion to uncoated cell-culture plastic is a hallmark of α_{M}β₂ integrin activity (Yakubenko et al., 2002). When THP-1 cells were cultured on plastic in the presence of 20 nM PBDu, they became strongly adherent and were resistant to washing with PBS (FIG. 5A). The chemicals had no effect on this activity. Interestingly, when the cells were washed with 2.5 mM EDTA, a significant portion of the IMB-10 treated cells resisted detachment and remained adherent (FIGS. 5A and B). The IMB-8 compound or a chemical gelatinase inhibitor (InhI) did not have such an effect. Increased resistance to detachment was also observed on fibrinogen substratum. Again, the chemicals had no measurable effect on PBS washing, but IMB-10 treated cells were more resistant to washings with EDTA (FIG. 5C). Similar results were obtained with OCI-AML-3 cells (not shown). In the absence of phorbol ester activation, IMB-10 did not increase cell adhesion indicating that it stabilizes the active I domain rather than activates it (data not shown).

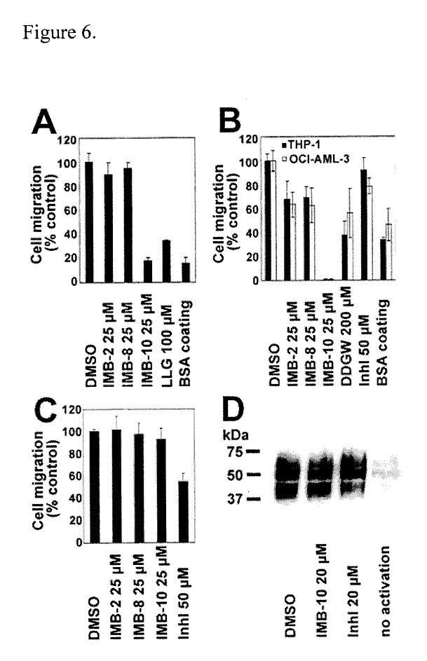

The effect of the compounds on cell migration was evaluated in a transwell assay. THP-1 cells migrate on a synthetic LLG-C4-GST peptide coating in a β₂ integrin dependent manner (Koivunen et al., 2001). Here, IMB-10 potently inhibited cell migration as did the LLG-C4 peptide (FIG. 6A). The less active compounds IMB-2 and -8 did not siginificantly inhibit cell migration at a 25 μM concentration. At a 100 μM concentration, IMB-2 also became inhibitory (not shown). Similar results were obtained by studying α_{M}β₂ integrin-dependent migration on fibrinogen-coated transwells. Again, IMB-10 completely inhibited migration of THP-1 and OCI-AML-3 cells at a 25 μM concentration and IMB-2 and -8 were less active (FIG. 6B). An inhibitory effect was also obtained by DDGW, but not by the gelatinase inhibitor InhI. The α_{M} I domain-binding chemicals did not have any effect on β₂ integrin-independent cell motility. Results for HT1080 fibrosarcoma cells are shown in FIG. 6C. The migration of these cells was partially inhibited with the gelatinase selective inhibitor InhI.

As proMMP-9 interacts with α_{M}β₂ integrin on the cell surface, we evaluated the effect of MBs on pericellular gelatinase activity. We have previously shown that pericellular proteolysis of urokinase-plasminogen activator (uPAR) is dependent on gelatinase activity. OCI-AML-3 cells stimulated with PDBu showed a high level of cleaved uPAR D2+3 form, which does not bind to the urokinase-plasminogen activator or α_{M}β₂ integrin. The cleavage of uPAR could not be inhibited with IMB-10 at a 20 μM concentration, whereas 20 μM gelatinase-selective inhibitor (InhI) reduced uPAR proteolysis indicating that the activated integrin does not prevent gelatinase-mediated proteolysis (FIG. 6D).

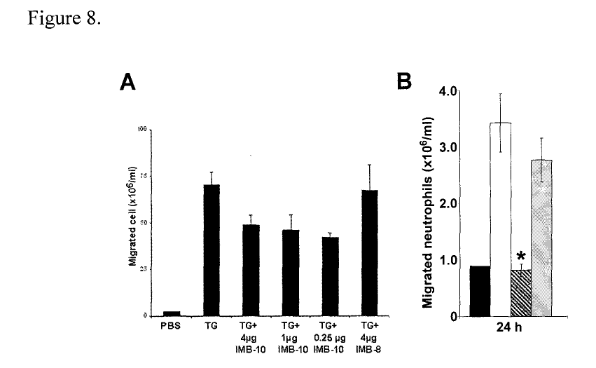

Suppression of inflammation in vivo. As leukocyte integrins are needed for proper inflammatory response, we tested the functionality of IMB-10 in thioglycollate-induced peritonitis in mice. In this model, the DDGW peptide potently inhibits the emigration of activated neutrophils into the peritoneal cavity (Stefanidakis et al., 2004). Intravenously injected IMB-10 showed a dose-dependent decrease in the number of leukocytes emigrated into the peritoneum three hours after induction of inflammation. IMB-8 did not inhibit neutrophil accumulation (FIG. 8A). After 24 hours the number of leukocytes was still low in the mice treated with IMB-10, but not by IMB-8 (FIG. 8A). Approximately 50-60% of the emigrated cells were neutrophils after 24 h, the rest of the cells consisting mainly of macrophages and T cells.

### REFERENCES

Arnaout, M. A., Goodman, S. L. and Xiong, J. P. (2002) Coming to grips with integrin binding to ligands. Curr Opin Cell Biol, 14: 641-51.

Bansal, V. S., Vaidya, S., Somers, E. P., Kanuga, M., Shevell, D., Weikel, R. and Detmers, P. A. (2003) Small molecule antagonists of complement receptor type 3 block adhesion and adhesion-dependent oxidative burst in human polymorphonuclear leukocytes. J Pharmacol Exp Ther, 304: 1016-24.

Beglova, N., Blacklow, S. C., Takagi, J. and Springer, T. A. (2002) Cysteine-rich module structure reveals a fulcrum for integrin rearrangement upon activation. Nat Struct Biol, 9: 282-7.

Bjorklund, M., Heikkila, P. and Koivunen, E. (2004) Peptide inhibition of catalytic and noncatalytic activities of matrix metalloproteinase-9 blocks tumor cell migration and invasion. J Biol Chem, 30: 30.

Goodman, S. L., Holzemann, G., Sulyok, G. A. and Kessler, H. (2002) Nanomolar small molecule inhibitors for alphav(beta)6, alphav(beta)5, and alphav(beta)3 integrins. J Med Chem, 45: 1045-51.

Hyde-DeRuyscher, R., Paige, L. A., Christensen, D. J., Hyde-DeRuyscher, N., Lim, A., Fredericks, Z. L., Kranz, J., Gallant, P., Zhang, J., Rocklage, S. M., Fowlkes, D. M., Wendler, P. A. and Hamilton, P. T. (2000) Detection of small-molecule enzyme inhibitors with peptides isolated from phage-displayed combinatorial peptide libraries. Chem Biol, 7: 17-25.

Hynes, R. O. (2002) Integrins: bidirectional, allosteric signaling machines. Cell, 110: 673-87.

Kallen, J., Welzenbach, K., Ramage, P., Geyl, D., Kriwacki, R., Legge, G., Cottens, S., Weitz-Schmidt, G. and Hommel, U. (1999) Structural basis for LFA-1 inhibition upon lovastatin binding to the CD11a I-domain. J Mol Biol, 292: 1-9.

Kay, B., Kurakin, A. and Hyde-DeRuyscher, R. (1998) From peptides to drugs via phage display. Drug Discovery Today, 3: 370-378.

Kelly, T. A., Jeanfavre, D. D., McNeil, D. W., Woska, J. R., Jr., Reilly, P. L., Mainolfi, E. A., Kishimoto, K. M., Nabozny, G. H., Zinter, R., Bormann, B. J. and Rothlein, R. (1999) Cutting edge: a small molecule antagonist of LFA-1-mediated cell adhesion. J Immunol, 163: 5173-7.

Koivunen, E., Arap, W., Valtanen, H., Rainisalo, A., Medina, O. P., Heikkila, P., Kantor, C., Gahmberg, C. G., Salo, T., Konttinen, Y. T., Sorsa, T., Ruoslahti, E. and Pasqualini, R. (1999) Tumor targeting with a selective gelatinase inhibitor. Nat Biotechnol, 17: 768-74.

Koivunen, E., Ranta, T. M., Annila, A., Taube, S., Uppala, A., Jokinen, M., van Willigen, G., Ihanus, E. and Gahmberg, C. G. (2001) Inhibition of beta(2) integrin-mediated leukocyte cell adhesion by leucine-leucine-glycine motif-containing peptides. J Cell Biol, 153: 905-16.

Last-Barney, K., Davidson, W., Cardozo, M., Frye, L. L., Grygon, C. A., Hopkins, J. L., Jeanfavre, D. D., Pav, S., Qian, C., Stevenson, J. M., Tong, L., Zindell,

R. and Kelly, T. A. (2001) Binding site elucidation of hydantoin-based antagonists of LFA-1 using multidisciplinary technologies: evidence for the allosteric inhibition of a protein—protein interaction. J Am Chem Soc, 123: 5643-50.

Li, R., Xie, J., Kantor, C., Koistinen, V., Altieri, D. C., Nortamo, P. and Gahmberg, C. G. (1995) A peptide derived from the intercellular adhesion molecule-2 regulates the avidity of the leukocyte integrins CD11b/CD18 and CD11c/CD18. J Cell Biol, 129: 1143-53.

Liu, G., Huth, J. R., Olejniczak, E. T., Mendoza, R., DeVries, P., Leitza, S., Reilly, E. B., Okasinski, G. F., Fesik, S. W. and von Geldern, T. W. (2001) Novel parylthio cinnamides as antagonists of leukocyte function-associated antigen-1/intracellular adhesion molecule-1 interaction. 2. Mechanism of inhibition and structure-based improvement of pharmaceutical properties. J Med Chem, 44: 1202-10.

Luo, B. H., Takagi, J. and Springer, T. A. (2004) Locking the beta3 integrin I-like domain into high and low affinity conformations with disulfides. J Biol Chem, 279: 10215-21. Epub 2003 Dec. 16.

McCleverty, C. J. and Liddington, R. C. (2003) Engineered allosteric mutants of the integrin alphMbeta2 I domain: structural and functional studies. Biochem J, 372: 121-7.

Michishita, M., Videm, V. and Arnaout, M. A. (1993) A novel divalent cation-binding site in the A domain of the beta 2 integrin CR3 (CD11b/CD18) is essential for ligand binding. Cell, 72: 857-67.

Palecek, S. P., Loftus, J. C., Ginsberg, M. H., Lauffenburger, D. A. and Horwitz, A. F. (1997) Integrin-ligand binding properties govern cell migration speed through cell-substratum adhesiveness. Nature, 385: 537-40.

Pasqualini, R., Koivunen, E., Kain, R., Lahdenranta, J., Sakamoto, M., Stryhn, A., Ashmun, R. A., Shapiro, L. H., Arap, W. and Ruoslahti, E. (2000) Aminopeptidase N is a receptor for tumor-homing peptides and a target for inhibiting angiogenesis. Cancer Res, 60: 722-7.

Ripka, A. S. and Rich, D. H. (1998) Peptidomimetic design. Curr Opin Chem Biol, 2: 441-52.

Rowsell, S., Hawtin, P., Minshull, C. A., Jepson, H., Brockbank, S. M., Barratt, D. G., Slater, A. M., McPheat, W. L., Waterson, D., Henney, A. M. and Pauptit, R. A. (2002) Crystal structure of human MMP9 in complex with a reverse hydroxamate inhibitor. J Mol Biol, 319: 173-81.

Ruoslahti, E. (1996) RGD and other recognition sequences for integrins. Annu Rev Cell Dev Biol, 12: 697-715.

Salas, A., Shimaoka, M., Kogan, A. N., Harwood, C., von Andrian, U. H. and Springer, T. A. (2004) Rolling adhesion through an extended conformation of integrin alphaLbeta2 and relation to alpha I and beta I-like domain interaction. Immunity, 20: 393-406.

Shimaoka, M., Salas, A., Yang, W., Weitz-Schmidt, G. and Springer, T. A. (2003) Small molecule integrin antagonists that bind to the beta2 subunit I-like domain and activate signals in one direction and block them in the other. Immunity, 19: 391-402.

Stefanidakis, M., Bjorklund, M., Ihanus, E., Gahmberg, C. G. and Koivunen, E. (2003) Identification of a negatively charged peptide motif within the catalytic domain of progelatinases that mediates binding to leukocyte beta 2 integrins. J Biol Chem, 278: 34674-84.

Stefanidakis, M., Ruohtula, T., Borregaard, N., Gahmberg, C. G. and Koivunen, E. (2004) Intracellular and cell surface localization of a complex between alphMbeta2 integrin and promatrix metalloproteinase-9 progelatinase in neutrophils. J Immunol, 172: 7060-8.

Weitz-Schmidt, G., Welzenbach, K., Brinkmann, V., Kamata, T., Kallen, J., Bruns, C., Cottens, S., Takada, Y. and Hommel, U. (2001) Statins selectively inhibit leukocyte function antigen-1 by binding to a novel regulatory integrin site. Nat Med, 7: 687-92.

Xiong, J. P., Li, R., Essafi, M., Stehle, T. and Arnaout, M. A. (2000) An isoleucine-based allosteric switch controls affinity and shape shifting in integrin CD11b A-domain. J Biol Chem, 275: 38762-7.

Xiong, J. P., Stehle, T., Diefenbach, B., Zhang, R., Dunker, R., Scott, D. L., Joachimiak, A., Goodman, S. L. and Arnaout, M. A. (2001) Crystal structure of the extracellular segment of integrin alpha Vbeta3. Science, 294: 339-45. Epub 2001 Sep. 6.

Xiong, J. P., Stehle, T., Zhang, R., Joachimiak, A., Frech, M., Goodman, S. L. and Arnaout, M. A. (2002) Crystal structure of the extracellular segment of integrin alpha Vbeta3 in complex with an Arg-Gly-Asp ligand. Science, 296: 151-5. Epub 2002 Mar. 7.

Yakubenko, V. P., Lishko, V. K., Lam, S. C. and Ugarova, T. P. (2002) A molecular basis for integrin alpha Mbeta 2 ligand binding promiscuity. J Biol Chem, 107:1.

1. A thioxothiazolidinone compound of formula I 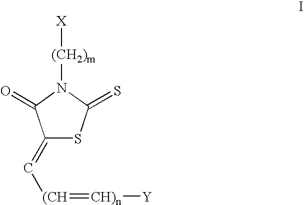
wherein m is 0, 1 or 2;
X is H, cycloalkyl or phenyl, which is unsubstituted or substituted with one or more substituents selected from the group consisting of lower alkyl, hydroxy and halogen;
n is 0 or 1;
Y is phenyl, furanyl, indole or pyrrole, which all may be substituted with one or more substituents selected from the group consisting of lower alkyl, lower alkoxy, halogen, (3,5-dimethylphenoxy)propoxy, and phenyl, wherein phenyl may be further substituted with one or more halogen atoms, nitro, amino or carboxyl groups; for use as a pharmaceutical.

2. A compound of formula I as defined in claim 1, wherein m is 0, X is unsubstituted phenyl or phenyl ortho-substituted with methyl, n is 1 and Y is unsubstituted phenyl.

3. A thioxothiazolidinone compound selected from the group consisting of (E)-5-(4-(3-(3,5-dimethylphenoxy)propoxy)-3-methoxybenzylidene)-3-ethyl-2-thioxothiazolidin-4-one (IMB-2);
(Z)-3-benzyl-5-((5-(3-nitrophenyl)furan-2-yl)methylene)-2-thioxothiazolidin-4-one (IMB-6); and
(5Z)-5((E)-3-phenylallylidene)-2-thioxo-3-o-tolylthiazolidin-4-one (IMB-10), for use as a pharmaceutical.

4. A pharmaceutical composition comprising a thioxothiazolidinone compound of the formula I 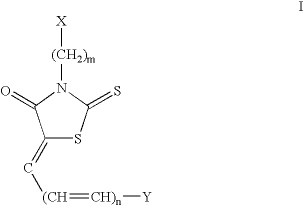
wherein m is 0, 1 or 2;
X is H, cycloalkyl or phenyl, which is unsubstituted or substituted with one or more substituents selected from the group consisting of lower alkyl, hydroxy and halogen;
n is 0 or 1;
Y is phenyl, furanyl, indole or pyrrole, which all may be substituted with one or more substituents selected from the group consisting of lower alkyl, lower alkoxy, halogen, (3,5-dimethylphenoxy)propoxy, and phenyl, wherein phenyl may be further substituted with one or more halogen atoms, nitro, amino or carboxyl groups; and a pharmaceutically acceptable carrier.

5. The pharmaceutical composition according to claim 4 wherein in the formula I m is 0, X is unsubstituted phenyl or phenyl ortho-substituted with methyl, n is 1 and Y is unsubstituted phenyl.

6. The pharmaceutical composition according to claim 4, wherein the thioxothiazolidinone compound is selected from the group consisting of
(E)-5-(4-(3-(3,5-dimethylphenoxy)propoxy)-3-methoxybenzylidene)-3-ethyl-2-thioxothiazolidin-4-one (IMB-2);
(Z)-3-benzyl-5-((5-(3-nitrophenyl)furan-2-yl)methylene)-2-thioxothiazolidin-4-one (IMB-6); and
(5Z)-5((E)-3-phenylallylidene)-2-thioxo-3-o-tolylthiazolidin-4-one (IMB-10).

7. Use of a thioxothiazolidinone compound having the formula 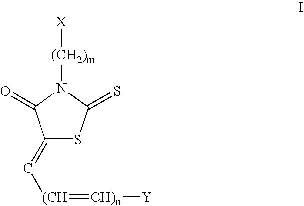
wherein m is 0, 1 or 2;
X is H, cycloalkyl or phenyl, which is unsubstituted or substituted with one or more substituents selected from the group consisting of lower alkyl, hydroxy and halogen;
n is 0 or 1;
Y is phenyl, furanyl, indole or pyrrole, which all may be substituted with one or more substituents selected from the group consisting of lower alkyl, lower alkoxy, halogen, (3,5-dimethylphenoxy)propoxy, and phenyl, wherein phenyl may be further substituted with one or more halogen atoms, nitro, amino or carboxyl groups;
for the manufacture of a pharmaceutical composition for the treatment of conditions dependent on leukocyte cell migration.

8. Use according to claim 7, wherein in the formula I m is 0, X is unsubstituted phenyl or phenyl ortho-substituted with methyl, n is 1 and Y is unsubstituted phenyl.

9. Use according to claim 7 wherein the conditions dependent on leukocyte cell migration are selected from the group consisting of leukaemia, other malignancies/cancers and inflammatory conditions.

10. Use according to any one of claims 7 to 9, wherein the thioxothiazolidinone compound is selected from the group consisting of
(E)-5-(4-(3-(3,5-dimethylphenoxy)propoxy)-3-methoxybenzylidene)-3-ethyl-2-thioxothiazolidin-4-one (=IMB-2);
(Z)-3-benzyl-5-((5-(3-nitrophenyl)furan-2-yl)methylene)-2-thioxothiazolidin-4-one (=IMB-6); and
(5Z)-5((E)-3-phenylallylidene)-2-thioxo-3-o-tolylthiazolidin-4-one (=IMB-10).

11. A method for therapeutic or prophylactic treatment of conditions dependent on leukocyte migration, comprising administering to a mammal in need of such treatment a thioxothiazolidinone compound of the formula 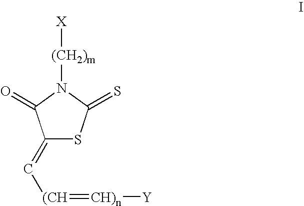
wherein m is 0, 1 or 2;
X is H, cycloalkyl or phenyl, which is unsubstituted or substituted with one or more substituents selected from the group consisting of lower alky, hydroxy and halogen;
n is 0 or 1;
Y is phenyl, furanyl, indole or pyrrole, which all may be substituted with one or more substituents selected from the group consisting of lower alky, lower alkoxy, halogen, (3,5-dimethylphenoxy)propoxy, and phenyl, wherein phenyl may be further substituted with one or more halogen atoms, nitro, amino or carboxyl groups;
in an amount which is effective in inhibiting leukocyte cell migration.

12. A method for therapeutic or prophylactic treatment of leukaemia and other malignancies/cancers, comprising administering to a mammal in need of such treatment a compound comprising a thioxothiazolidinone compound of the formula 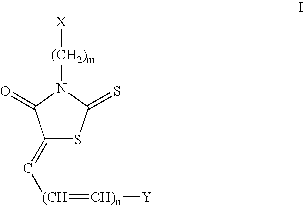
wherein m is 0, 1 or 2;
X is H, cycloalkyl or phenyl, which is unsubstituted or substituted with one or more substituents selected from the group consisting of lower alky, hydroxy and halogen;
n is 0 or 1;
Y is phenyl, furanyl, indole or pyrrole, which all may be substituted with one or more substituents selected from the group consisting of lower alkyl, lower alkoxy, halogen, (3,5-dimethylphenoxy)propoxy, and phenyl, wherein phenyl may be further substituted with one or more halogen atoms, nitro, amino or carboxyl groups;
in an amount which is effective in the treatment of leukaemia and other malignancies/cancers.

13. A method for therapeutic or prophylactic treatment of inflammatory conditions, comprising administering to a mammal in need of such treatment a compound comprising a thioxothiazolidinone compound of the formula I 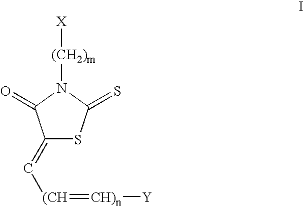
wherein m is 0, 1 or 2;
X is H, cycloalkyl or phenyl, which is unsubstituted or substituted with one or more substituents selected from the group consisting of lower alkyl, hydroxy and halogen; p1 n is 0 or 1;
Y is phenyl, furanyl, indole or pyrrole, which all may substituted with one or more substituents selected from the group consisting of lower alkyl, lower alkoxy, halogen, (3,5-dimethylphenoxy)propoxy, and phenyl, wherein phenyl may be further substituted with one or more halogen atoms, nitro, amino or carboxyl groups;
in an amount which is effective in the treatment of inflammatory conditions.

14. The method according to any one of claims 11 to 13, wherein the thioxothiazolidinone compound is selected from the group consisting of
(E)-5-(4-(3-(3,5-dimethylphenoxy)propoxy)-3-methoxybenzylidene)-3-ethyl-2-thioxothiazolidin-4-one (=IMB-2);
(Z)-3-benzyl-5-((5-(3-nitrophenyl)furan-2-yl)methylene)-2-thioxothiazolidin-4-one (=IMB-6); and
(5Z)-5((E)-3-phenylallylidene)-2-thioxo-3-o-tolylthiazolidin-4-one (=IMB-10).
